(19) United States
(12) Patent Application Publication
(10) Pub. No.: US 2022/0257580 A1
(43) Pub. Date: Aug. 18, 2022

(54) TREATMENT OF C3 GLOMERULOPATHY USING A C5A INHIBITOR

(71) Applicant: CHEMOCENTRYX, INC., San Carlos, CA (US)

(72) Inventor: Thomas J. SCHALL, San Carlos, CA (US)

(21) Appl. No.: 17/556,188

(22) Filed: Dec. 20, 2021

Related U.S. Application Data
(60) Provisional application No. 63/128,397, filed on Dec. 21, 2020.

(51) Int. Cl. A61K 31/451; A61P 13/12

(52) U.S. Cl. CPC A61K 31/451; A61P 13/12

(57) ABSTRACT

Methods of treating certain human patient populations suffering from or susceptible to C3 glomerulopathy comprising administering to the human an effective amount of a C5aR antagonist are provided.

### CROSS-REFERENCES TO RELATED APPLICATIONS

This application claims the benefit of priority under 35 U.S.C. § 119(e) to U.S. Provisional Application Ser. No. 63/128,397 filed Dec. 21, 2020, the contents of which is herein incorporated by reference in its entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

C3 glomerulopathy (C3G) is a rare disease of the kidney (the prevalence of C3G is estimated at 2-3 per 1,000,000 people). C3G is characterized by deposition of the protein known as C3 (a component of the body's complement system) in the filtration units (the glomeruli) of the kidney, indicating complement involvement in causing kidney damage. C3 glomerulopathy is characterized by evidence of alternative complement activation based on C3 deposition in the glomeruli. There are two forms of the disease: dense deposit disease (DDD, formerly called membranoproliferative glomerulonephritis [MPGN] Type II) and C3 glomerulonephritis (C3GN, formerly called idiopathic MPGN). Genetic lesions leading to defective complement regulation, including mutations in complement factor H (CFH) have been described in these patients. Patients with C3 glomerulopathy often have high proteinuria and progressive deterioration in renal function. There is no approved treatment for patients with C3 glomerulopathy, including C3GN. Without treatment, C3G invariably leads to kidney failure, and kidney transplant is frequently the only option. Even after transplantation, the new kidney will frequently fail due to recurrence of the disease.

### BRIEF SUMMARY OF THE INVENTION

The present disclosure is directed to a method of treating certain human patient populations suffering from or susceptible to complement 3 (C3) glomerulopathy comprising administering to the human an effective amount of a C5aR antagonist of Formula I

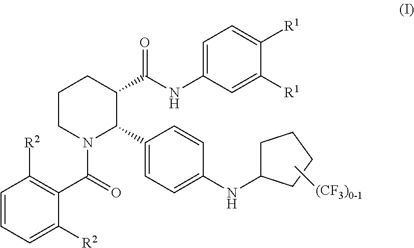

or a pharmaceutically acceptable salt thereof. In some embodiments said therapeutically effective amount is about 10 mg or 30 mg of the compound twice daily. In some embodiments,
each R¹ is independently selected from the group consisting of CH₃, CF₃, CH₂CH₃, Cl, 1-pyrrolidine, ˜O—CH(CH₃)₂, and CH₂OH; and
   each R² is independently selected from the group consisting of CH₃ and F.

In some embodiments, the C5aR antagonist is a compound having the formula:

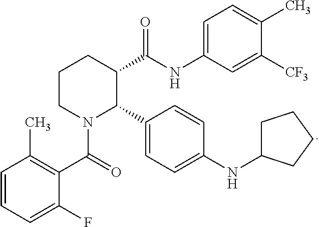 [SMILES: Cc1ccc(NC(=O)[C@H]2CCCN(C(=O)c3c(C)cccc3F)[C@H]2c2ccc(NC3CCCC3)cc2)cc1C(F)(F)F]

In some embodiments, the C5aR antagonist is a compound having the formula:

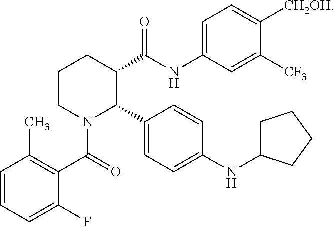 [SMILES: Cc1cccc(F)c1C(=O)N1CCC[C@H](C(=O)Nc2ccc(CO)c(C(F)(F)F)c2)[C@@H]1c1ccc(NC2CCCC2)cc1]

### BRIEF DESCRIPTION OF THE DRAWINGS

FIG. 1 represents the patient's Estimated glomerular filtration rate (eGFR) before and after treatment with compound 1.

FIG. 2 represents the histopathological improvement following treatment with compound 1.

### DETAILED DESCRIPTION OF THE INVENTION

Abbreviation and Definitions

As used herein, the term “treating” or “treatment” encompasses both disease-modifying treatment and symptomatic treatment, either of which may be prophylactic (i.e., before the onset of symptoms, in order to prevent, delay or reduce the severity of symptoms) or therapeutic (i.e., after the onset of symptoms, in order to reduce the severity and/or duration of symptoms). Treatment methods provided herein include, in general, administration to a patient an effective amount of one or more compounds provided herein. Suitable patients include those patients suffering from or susceptible to {i.e., prophylactic treatment) a disorder or disease identified herein. Typical patients for treatment as described herein include mammals, particularly primates, especially humans. Other suitable patients include domesticated companion animals such as a dog, cat, horse, and the like, or a livestock animal such as cattle, pig, sheep and the like.

The term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

Compound 1 (avacopan) has the formula:

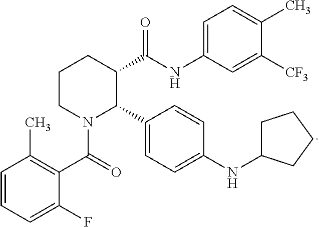 [SMILES: Cc1ccc(NC(=O)[C@H]2CCCN(C(=O)c3c(C)cccc3F)[C@H]2c2ccc(NC3CCCC3)cc2)cc1C(F)(F)F]

The compounds described in the Embodiments below can be obtained according to methods described in WO 2010/075257, WO 2011/163640, WO 2016/053890.

### Embodiments

A. Methods of Treatment

The present disclosure is directed to methods of treating certain human patient populations suffering from or susceptible to complement 3 (C3) glomerulopathy comprising administering to the human an effective amount of a compound of Formula (I), or a pharmaceutically acceptable salt thereof,

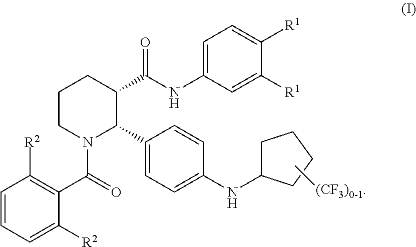

In some embodiments, therapeutically effective amount is about 10 mg or 30 mg of the compound twice daily. In some embodiments,
each R¹ is independently selected from the group consisting of CH₃, CF₃, CH₂CH₃, Cl, 1-pyrrolidine, ˜O—CH(CH₃)₂, and CH₂OH; and
   each R² is independently selected from the group consisting of CH₃ and F.

Certain human subpopulations of subjects can respond surprisingly well to treatment with compounds of Formula I. Unexpectedly, subjects receiving a compound of Formula I show a robust and significant improvement in eGFR after 26 weeks of therapy. The observed increase in eGFR in patients receiving a compound of Formula I was surprisingly high in such a low time frame (26 weeks). In some embodiments, subjects with an eGFR of <60 mL/min/1.73 m² at baseline responded significantly well relative to placebo. It is understood that subjects receiving a placebo are untreated (i.e., not receiving an effective amount of a compound of Formula I). In some embodiments, the change in eGFR from baseline to after week 26 in subjects receiving a compound of Formula I is at least a 5% improvement. In some embodiments, the change in eGFR from baseline to after week 26 in subjects receiving a compound of Formula I is at least a 10% improvement. In some embodiments, the change in eGFR from baseline to after week 26 in subjects receiving a compound of Formula I is about a 13% improvement. In some embodiments, the average change in eGFR from baseline to after week 26 in subjects receiving a compound of Formula I is about a 5% improvement. Comparatively, in some embodiments, the change in eGFR from baseline to after week 26 in subjects receiving placebo is at least a 5% worsening. In some embodiments, the average change in eGFR from baseline to after week 26 in subjects receiving placebo is about a 6% worsening.

In some embodiments, subjects receiving a compound of Formula I show a robust improvement in urinary MCP-1:Creatinine ratio after 26 weeks of therapy as compared to placebo. In some embodiments, the change in urinary MCP-1:Creatinine ratio from baseline to after week 26 in subjects receiving a compound of Formula I is at least a 5% improvement. In some embodiments, the change in urinary MCP-1:Creatinine ratio from baseline to after week 26 in subjects receiving a compound of Formula I is at least a 10% improvement. In some embodiments, the change in urinary MCP-1:Creatinine ratio from baseline to after week 26 in subjects receiving a compound of Formula I is about a 12% improvement. Comparatively, in some embodiments, the change in urinary MCP-1:Creatinine ratio from baseline to after week 26 in subjects receiving placebo (i.e., not receiving an effective amount of a compound of Formula I) is no change or about a 1% worsening.

In some embodiments, subjects receiving a compound of Formula I show a robust improvement in urinary Protein:Creatinine ratio after 26 weeks of therapy as compared to placebo. In some embodiments, the change in urinary Protein:Creatinine ratio from baseline to after week 26 in subjects receiving a compound of Formula I is at least a 15% improvement. In some embodiments, the change in urinary Protein:Creatinine ratio from baseline to after week 26 in subjects receiving a compound of Formula I is at least a 20% improvement. In some embodiments, the change in urinary Protein:Creatinine ratio from baseline to after week 26 in subjects receiving a compound of Formula I is about a 26% improvement. Comparatively, in some embodiments, the change in urinary Protein:Creatinine ratio from baseline to after week 26 in subjects receiving placebo is about a 14% improvement.

In some embodiments, subjects receiving a compound of Formula I show a robust improvement in average C3G Histologic Index (CHI) after 26 weeks of therapy as compared to placebo. In some embodiments, the CHI from baseline to after week 26 in subjects receiving a compound of Formula I is about the same or about a 5% improvement. In some embodiments, the change in CHI from baseline to after week 26 in subjects receiving a compound of Formula I is an average of about a 6% improvement. In some embodiments, the change in CHI from baseline to after week 26 in subjects receiving placebo is at least a 20% worsening. In some embodiments, the change in CHI from baseline to after week 26 in subjects receiving placebo is an average of about a 26% worsening. Methods for measuring CHI are described in Bomback, et al. C3 glomerulonephritis and dense deposit disease share a similar disease course in a large United States cohort of patients with C3 glomerulopathy. Kidney Int. 2018 93(4):977-985.

In some embodiments, subjects with high baseline (before treatment) C5b-9 plasma levels responded significantly better to treatment as compared to subjects with low baseline (before treatment) C5b-9 plasma levels. High baseline C5b-9 plasma levels can include subjects who have 50%, 75% or more C5b-9 in their blood plasma as compared to a threshold value (the average C59b plasma levels for healthy individuals who are not diagnosed with C3G). Low baseline C5b-9 levels includes subjects who have C5b-9 plasma levels that are lower than the established high baseline C5b-9 plasma levels. In some embodiments, the average C5b-9 plasma level for healthy individuals is ˜150 ng/mL. In some embodiments, high C5b-9 baseline plasma levels refers to subjects with a blood plasma concentration of >244 ng/mL. In some embodiments, low C5b-9 baseline plasma levels refers to subjects with ≤244 ng/mL. In some embodiments, subjects with high baseline C5b-9 plasma levels demonstrate a statistically significant improvement in a clinical metric for measuring C3G disease progression, whereas those with low baseline C5b-9 plasma levels do not.

In some embodiments, subjects with high baseline (before treatment) C3, C3d, C3c, C3adesArg, or C4 plasma levels responded significantly better to treatment as compared to subjects with low baseline (before treatment) C3, C3d, C3c, C3adesArg, or C4 plasma levels. High baseline C3, C3d, C3c, C3adesArg, or C4 plasma levels can include subjects who have 20%, 50% or more of the subject protein in their blood plasma as compared to a threshold value (the average plasma levels of the subject protein for healthy individuals who are not diagnosed with C3G). Low baseline C3, C3d, C3c, C3adesArg, or C4 levels include subjects whose baseline subject protein plasma levels are lower than the established high baseline protein plasma levels. In some embodiments, subjects with high baseline C3, C3d, C3c, C3adesArg, or C4 plasma levels demonstrate a statistically significant improvement in a clinical metric for measuring C3G disease progression, whereas those with low baseline C3, C3d, C3c, C3adesArg, or C4 baseline plasma levels do not. In some embodiments, the average C3 level for a healthy individual is about 125 mg/dL. In some embodiments, the average C4 level for a healthy individual is about 30 mg/dL.

In some embodiments, subjects with high baseline (before treatment) C3 nephritic factor plasma levels responded significantly better to treatment as compared to subjects with low baseline (before treatment) C3 nephritic factor plasma levels. High baseline C3 nephritic factor plasma levels can include subjects who have 20%, 50% or more C3 nephritic factor in their blood plasma as compared to a threshold value (the average C3 nephritic factor plasma levels for healthy individuals who are not diagnosed with C3G). Low baseline C3 nephritic factor levels includes subjects who have C3 nephritic factor plasma levels that are lower than the established high baseline C3 nephritic factor plasma levels. In some embodiments, subjects with high baseline C3 nephritic factor plasma levels demonstrate a statistically significant improvement in a clinical metric for measuring C3G disease progression, whereas those with low baseline C3 nephritic factor baseline plasma levels do not.

In some embodiments, subjects with high baseline (before treatment) C5, C5a, C5b-9, or C5adesArg plasma levels responded significantly better to treatment as compared to subjects with low baseline (before treatment) C5, C5a, C5b-9, or C5adesArg plasma levels. High baseline C5, C5a, C5b-9, or C5adesArg plasma levels can include subjects who have 20%, 50% or more of the subject protein in their blood plasma as compared to a threshold value (the average plasma levels of the subject protein for healthy individuals who are not diagnosed with C3G). Low baseline C5, C5a, C5b-9, or C5adesArg levels include subjects whose baseline subject protein plasma levels are lower than the established high baseline protein plasma levels. In some embodiments, subjects with high baseline C5, C5a, C5b-9, or C5adesArg plasma levels plasma levels demonstrate a statistically significant improvement in a clinical metric for measuring C3G disease progression, whereas those with low baseline C5, C5a, C5b-9, or C5adesArg plasma levels baseline plasma levels do not.

In some embodiments, subjects with high baseline (before treatment) Serum Complement Factor H or Serum Complement Factor B plasma levels responded significantly better to treatment as compared to subjects with low baseline (before treatment) Serum Complement Factor H or Serum Complement Factor B plasma levels. High baseline Serum Complement Factor H or Serum Complement Factor B plasma levels can include subjects who have 20%, 50% or more of the subject protein in their blood plasma as compared to a threshold value (the average plasma levels of the subject protein for healthy individuals who are not diagnosed with C3G). Low baseline Serum Complement Factor H or Serum Complement Factor B levels include subjects whose baseline subject protein plasma levels are lower than the established high baseline protein plasma levels. In some embodiments, subjects with high baseline Serum Complement Factor H or Serum Complement Factor B plasma levels plasma levels demonstrate a statistically significant improvement in a clinical metric for measuring C3G disease progression, whereas those with low baseline Serum Complement Factor H or Serum Complement Factor B plasma levels baseline plasma levels do not.

In some embodiments, subjects with high baseline (before treatment) Serum Paraprotein plasma levels responded significantly better to treatment as compared to subjects with low baseline (before treatment) Serum Paraprotein plasma levels. High baseline Serum Paraprotein plasma levels can include subjects who have 20%, 50% or more Serum Paraprotein in their blood plasma as compared to a threshold value (the average Serum Paraprotein plasma levels for healthy individuals who are not diagnosed with C3G). Low baseline Serum Paraprotein levels include subjects whose baseline Paraprotein plasma levels are lower than the established high baseline protein plasma levels. In some embodiments, subjects with high baseline Serum Paraprotein plasma levels plasma levels demonstrate a statistically significant improvement in a clinical metric for measuring C3G disease progression, whereas those with low baseline Serum Paraprotein plasma levels baseline plasma levels do not.

In some embodiments, subjects with high baseline (before treatment) Complement Factor H Related Protein 5 (CFHR5) plasma levels responded significantly better to treatment as compared to subjects with low baseline (before treatment) CFHR5 plasma levels. High baseline CFHR5 plasma levels can include subjects who have 20%, 50% or more CFHR5 in their blood plasma as compared to a threshold value (the average CFHR5 plasma levels for healthy individuals who are not diagnosed with C3G). Low baseline CFHR5 levels include subjects whose baseline subject CFHR5 levels are lower than the established high baseline protein plasma levels. In some embodiments, subjects with high baseline CFHR5 plasma levels plasma levels demonstrate a statistically significant improvement in a clinical metric for measuring C3G disease progression, whereas those with low baseline CFHR5 plasma levels baseline plasma levels do not.

In some embodiments, subjects who responded surprisingly well to treatment are human patient populations with a complement protein plasma level baseline that is 20, 25%, or more above the average plasma levels for the complement protein in healthy individuals who are not diagnosed with C3G. In some embodiments, subjects who responded surprisingly well to treatment are human patient populations with a complement protein plasma level baseline that is 20, 25%, or more below the average plasma levels for the complement protein in healthy individuals who are not diagnosed with C3G. In some embodiments, the complement protein is C2, C3, C3d, C3c, C3adesArg, C4, C5a, C5b-9, or C5adesArg. In some embodiments, the average C2 level for a healthy individual is about 35 mg/dL.

When measuring patient response, a variety of clinical metrics can be used. For example, significant improvements can be observed by measuring one of more of the following metrics: percent change in the C3G Histologic Index (CHI) for disease activity and chronicity, percent change in Estimated Glomerular Filtration Rate (eGFR), percent change in the first morning urinary albumin:creatinine ratio (ACR), percent change in the first morning urinary protein:creatinine ratio (PCR), percent change in urinary MCP-1:creatinine ratio, percent change in EuroQOL-5D-5L (EQ-5D-5L), and percent change in Short Form-36 version 2 (SF-36 v2).

As measured by percent change in the C3G Histologic Index (CHI) for disease activity and chronicity, populations who respond significantly better to treatment include those where the proportion of subjects achieving at least 30% reduction in CHI is at least 5, 10, 15, 20, or 25% or more than the proportion of subjects who are not in the defined population. Methods for measuring CHI are described in Bomback, et al. C3 glomerulonephritis and dense deposit disease share a similar disease course in a large United States cohort of patients with C3 glomerulopathy. Kidney Int. 2018 93(4):977-985.

As measured by percent change in the Estimated Glomerular Filtration Rate (eGFR), populations who respond significantly better to treatment include those where the proportion of subjects achieving at least 20% improvement in eGFR is at least 5, 10, 15, 20, or 25% or more than the proportion of subjects who are not in the defined population.

As measured by percent change in the first morning urinary albumin:creatinine ratio (ACR), populations who respond significantly better to treatment include those where the proportion of subjects achieving at least 20% reduction in PCR is at least 5, 10, 15, 20, or 25% or more than the proportion of subjects who are not in the defined population.

As measured by percent change in the first morning urinary protein:creatinine ratio (PCR), populations who respond significantly better to treatment include those where the proportion of subjects achieving at least 20% reduction in PCR is at least 5, 10, 15, 20, or 25% or more than the proportion of subjects who are not in the defined population.

As measured by percent change in urinary MCP-1:creatinine ratio, populations who respond significantly better to treatment include those where the proportion of subjects achieving at least 20% reduction in urinary MCP-1:creatinine ratio is at least 5, 10, 15, 20, or 25% or more than the proportion of subjects who are not in the defined population.

The observed clinical changes in populations or subpopulations can vary depending on the timeframe for comparison. In some embodiments, the comparison in the preceding paragraphs is the change from baseline to Week 2. In some embodiments, the comparison in the preceding paragraphs is the change from baseline to Week 4. In some embodiments, the comparison in the preceding paragraphs is the change from baseline to Week 8. In some embodiments, the comparison in the preceding paragraphs is the change from baseline to Week 12. In some embodiments, the comparison in the preceding paragraphs is the change from baseline to Week 16. In some embodiments, the comparison in the preceding paragraphs is the change from baseline to Week 20. In some embodiments, the comparison in the preceding paragraphs is the change from baseline to Week 24. In some embodiments, the comparison in the preceding paragraphs is the change from baseline to Week 26. In some embodiments, the comparison in the preceding paragraphs is the change from baseline to Week 28. In some embodiments, the comparison in the preceding paragraphs is the change from baseline to Week 32. In some embodiments, the comparison in the preceding paragraphs is the change from baseline to Week 36. In some embodiments, the comparison in the preceding paragraphs is the change from baseline to Week 44.

In some embodiments, the complement 3 glomerulopathy is refractory to treatment. In some embodiments, the complement 3 glomerulonephritis is refractory to other treatment. In some embodiments, the human has refractory disease to immunosuppressive drugs. In some embodiments, the human has refractory disease to one or more of rituximab, cyclophosphamide, mycophenolate mofetil, tacrolimus, and steroids. In some embodiments, the human has refractory disease to one or more of rituximab, cyclophosphamide, mycophenolate mofetil, tacrolimus, and glucocorticosteroids.

B. Compounds of Formula I

Compounds of Formula (I), or a pharmaceutically acceptable salt thereof, have the structure

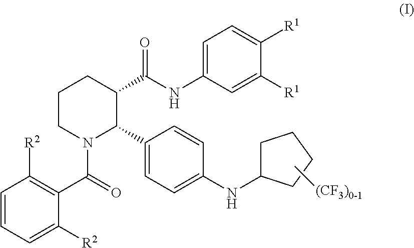

wherein
each R¹ is independently selected from the group consisting of CH₃, CF₃, CH₂CH₃, Cl, 1-pyrrolidine, ˜O—CH(CH₃)₂, and CH₂OH; and
each R² is independently selected from the group consisting of CH₃ and F.

In some embodiments, the compound of Formula I has the formula

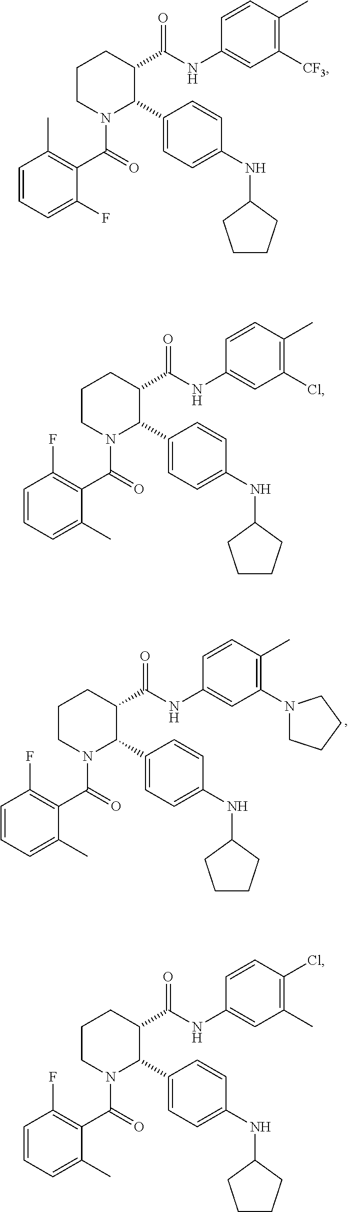 [SMILES: Cc1ccc(NC(=O)[C@H]2CCCN(C(=O)c3c(C)cccc3F)[C@H]2c2ccc(NC3CCCC3)cc2)cc1C(F)(F)F ; Cc1ccc(NC(=O)[C@H]2CCCN(C(=O)c3c(C)cccc3F)[C@H]2c2ccc(NC3CCCC3)cc2)cc1Cl ; Cc1ccc(NC(=O)[C@H]2CCCN(C(=O)c3c(C)cccc3F)[C@H]2c2ccc(NC3CCCC3)cc2)cc1N1CCCC1 ; Cc1cc(NC(=O)[C@H]2CCCN(C(=O)c3c(C)cccc3F)[C@H]2c2ccc(NC3CCCC3)cc2)ccc1Cl]

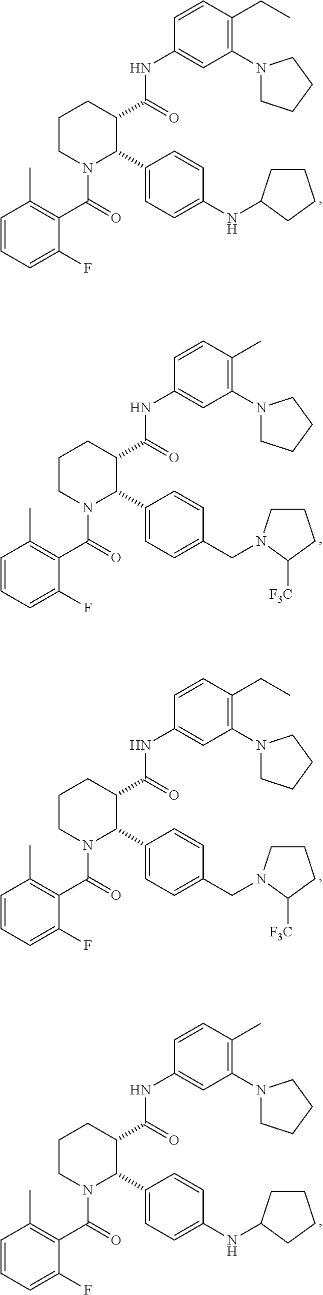 [SMILES: CCc1ccc(NC(=O)[C@H]2CCCN(C(=O)c3c(C)cccc3F)[C@H]2c2ccc(NC3CCCC3)cc2)cc1N1CCCC1 ; Cc1ccc(NC(=O)[C@H]2CCCN(C(=O)c3c(C)cccc3F)[C@H]2c2ccc(CN3CCCC3C(F)(F)F)cc2)cc1N1CCCC1 ; CCc1ccc(NC(=O)[C@H]2CCCN(C(=O)c3c(C)cccc3F)[C@H]2c2ccc(CN3CCCC3C(F)(F)F)cc2)cc1N1CCCC1 ; Cc1ccc(NC(=O)[C@H]2CCCN(C(=O)c3c(C)cccc3F)[C@H]2c2ccc(NC3CCCC3)cc2)cc1N1CCCC1]

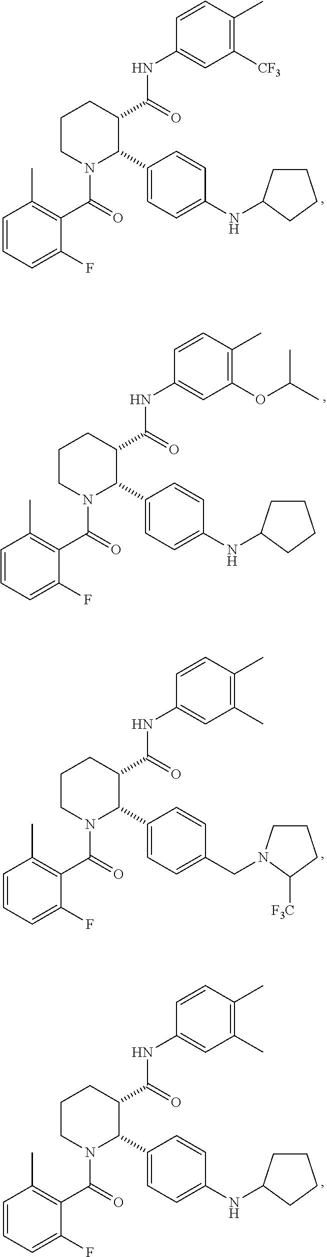 [SMILES: Cc1ccc(NC(=O)[C@H]2CCCN(C(=O)c3c(C)cccc3F)[C@H]2c2ccc(NC3CCCC3)cc2)cc1C(F)(F)F ; Cc1ccc(NC(=O)[C@H]2CCCN(C(=O)c3c(C)cccc3F)[C@H]2c2ccc(NC3CCCC3)cc2)cc1OC(C)C ; Cc1ccc(NC(=O)[C@H]2CCCN(C(=O)c3c(C)cccc3F)[C@H]2c2ccc(CN3CCCC3C(F)(F)F)cc2)cc1C ; Cc1ccc(NC(=O)[C@H]2CCCN(C(=O)c3c(C)cccc3F)[C@H]2c2ccc(NC3CCCC3)cc2)cc1C]

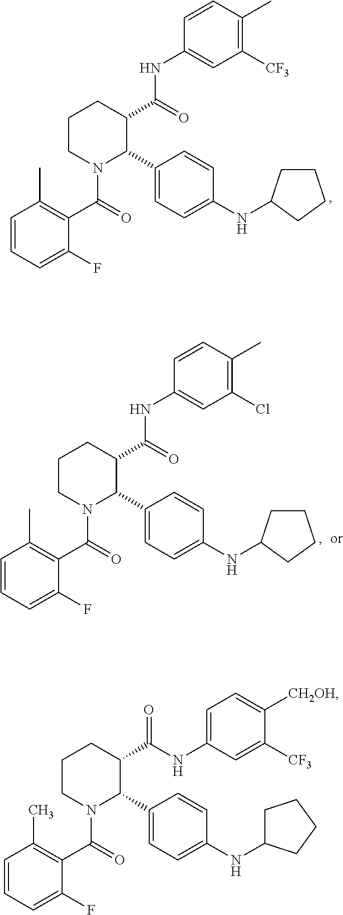 [SMILES: Cc1ccc(NC(=O)[C@H]2CCCN(C(=O)c3c(C)cccc3F)[C@H]2c2ccc(NC3CCCC3)cc2)cc1C(F)(F)F ; Cc1ccc(NC(=O)[C@H]2CCCN(C(=O)c3c(C)cccc3F)[C@H]2c2ccc(NC3CCCC3)cc2)cc1Cl ; Cc1cccc(F)c1C(=O)N1CCC[C@H](C(=O)Nc2ccc(CO)c(C(F)(F)F)c2)[C@@H]1c1ccc(NC2CCCC2)cc1]

or a pharmaceutically acceptable salt thereof

In some embodiments, the compound of Formula I has the formula

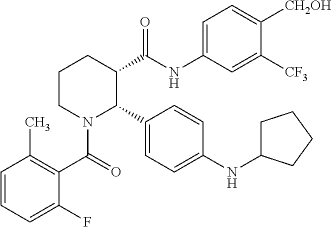 [SMILES: Cc1cccc(F)c1C(=O)N1CCC[C@H](C(=O)Nc2ccc(CO)c(C(F)(F)F)c2)[C@@H]1c1ccc(NC2CCCC2)cc1]

or a pharmaceutically acceptable salt thereof.

In some embodiments, the compound of Formula I is Compound 1, having the formula

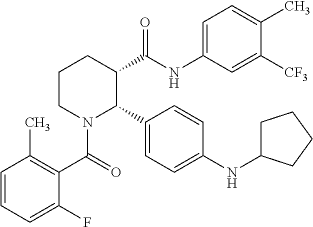 [SMILES: Cc1ccc(NC(=O)[C@H]2CCCN(C(=O)c3c(C)cccc3F)[C@H]2c2ccc(NC3CCCC3)cc2)cc1C(F)(F)F]

or a pharmaceutically acceptable salt thereof.

The compounds of Formula (I) described herein can be obtained according to methods described in WO 2010/075257, WO 2011/163640 and WO 2016/053890, the contents of each is hereby incorporated by reference for all purposes. In some embodiments, the compound of Formula (I) is a compound described in one of these references.

C. Methods of Administration

In general, treatment methods provided herein comprise administering to a patient an effective amount of a compound in specific dosages and timing to effectively treat C3 glomerulopathy. In some embodiments, the compound is administered to a subject (e.g., a human) orally. Treatment regimens may vary depending on the compound used and the route of administration, but a frequency of administration of 4 times daily or less is preferred. In some embodiments, a dosage regimen of 2 times daily is used. In some embodiments, a dosage regimen of 1 time daily is used.

The amount of time the individual receives treatment will depend on a variety of factors including the disease being threated as well as the age, body weight, general health, sex, diet, time of administration, and route of administration of the compound. In some embodiments, the subject receives treatment for 12 weeks. In some embodiments, the subject receives treatment for 26 weeks. In some embodiments, the subject receives treatment for 52 weeks. In some embodiments, the subject receives chronic treatment.

In some embodiments, the subject is orally administered 10 mg of Compound 1 twice daily, for a total daily dose of 20 mg.

In some embodiments, the subject is orally administered 15 mg of Compound 1 twice daily, for a total daily dose of 30 mg.

In some embodiments, the subject is orally administered 20 mg of Compound 1 twice daily, for a total daily dose of 40 mg.

In some embodiments, the subject is orally administered 25 mg of Compound 1 twice daily, for a total daily dose of 50 mg.

In some embodiments, the subject is orally administered 30 mg of Compound 1 twice daily, for a total daily dose of 60 mg.

D. Pharmaceutical Compositions

The compounds provided herein can be administered as compositions which will typically contain a pharmaceutical carrier or diluent.

The term “composition” as used herein is intended to encompass a product comprising the specified ingredients in the specified amounts, as well as any product which results, directly or indirectly, from combination of the specified ingredients in the specified amounts.

In some embodiments, the pharmaceutical composition further comprises one or more additional therapeutic agents.

The pharmaceutical compositions for the administration of the compounds of this disclosure may conveniently be presented in unit dosage form and may be prepared by any of the methods well known in the art of pharmacy and drug delivery. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions and self-emulsifications as described in U.S. Patent Application 2002-0012680, hard or soft capsules, syrups, elixirs, solutions, buccal patch, oral gel, chewing gum, chewable tablets, effervescent powder and effervescent tablets. Compositions intended for oral use may be prepared according to any method known to the art for the manufacture of pharmaceutical compositions and such compositions may contain one or more agents selected from the group consisting of sweetening agents, flavoring agents, coloring agents, antioxidants and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and U.S. Pat. No. 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, polyethylene glycol (PEG) of various average sizes (e.g., PEG400, PEG4000) and certain surfactants such as cremophor or solutol, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono- or di-glycerides, PEG esters and the like.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. Oral solutions can be prepared in combination with, for example, cyclodextrin, PEG and surfactants.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present disclosure may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols. Additionally, the compounds can be administered via ocular delivery by means of solutions or ointments. Still further, transdermal delivery of the subject compounds can be accomplished by means of iontophoretic patches and the like. For topical use, creams, ointments, jellies, solutions or suspensions, etc., containing the compounds of the present disclosure are employed. As used herein, topical application is also meant to include the use of mouth washes and gargles.

The compounds of this disclosure may also be coupled a carrier that is a suitable polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the compounds of the disclosure may be coupled to a carrier that is a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like. In one embodiment of the disclosure, the compound of the disclosure is coupled to a polymer or semipermeable polymer matrix that is formed as a stent or stent-graft device.

In some embodiments, the compounds provided herein are formulated as solid solution capsules as described in WO2020/112961, the contents of which is herein incorporated by reference for all purposes.

E. Combination Therapy

In some embodiments, the method further comprises administering to the human a therapeutically effective amount of one or more additional therapeutic agents. In some embodiments, the one or more additional therapeutic agents is administered sequentially or concurrently in the same composition or not.

In some embodiments, the one or more additional therapeutic agents is selected from immunosuppressive drugs, angiotensin-converting enzyme (ACE) inhibitors, angiotensin II type-1 receptor blockers (ARBs) and corticosteroids.

In some embodiments, the one or more additional therapeutic agents is selected from the group consisting of cyclophosphamide, mycophenolate mofetil, rituximab, eculizumab, tacrolimus, belimumab, OMS721, ACH-4471, AMY-101, Acthar Gel, SAND-5, corticotropin, CDX-1135, ramipril, perindopril, lisinopril, perindopril arginine, captopril, spirapril, quinapril, enalapril, imidapril, fosinopril, zofenopril, benazepril, trandolapril, verapamil, benazepril, amlodipine, trandolapril, P-003, cilazapril, delapril, moexipril, quinapril, fosinopril, temocapril, losartan, candesartan, irbesartan, telmisartan, olmesartan, valsartan, azilsartan, telmisartan, fimasartan, EMA-401, azilsartan medoxomil potassium, sparsentan, candesartan cilexetil, olmesartan medoxomil, TRV-027, losartan potassium, YH-22189, azilsartan trimethylethanolamine, allisartan isoproxil, and eprosartan. In some embodiments, the one or more additional therapeutic agents is selected from the group consisting of cyclophosphamide, mycophenolate mofetil, rituximab, eculizumab, and tacrolimus.

In some embodiments, the one or more additional therapeutic agents is selected from the group consisting of corticosteroids, steroids, immunosuppressants, Immunoglobulin G agonists, Dipeptidyl peptidase IV inhibitors, Lymphocyte function antigen-3 receptor antagonists, Interleukin-2 ligands, Interleukin-1 beta ligand inhibitors, IL-2 receptor alpha subunit inhibitors, HGF gene stimulators, IL-6 antagonists, IL-5 antagonists, Alpha 1 antitrypsin stimulators, Cannabinoid receptor antagonists, Histone deacetylase inhibitors, AKT protein kinase inhibitors, CD20 inhibitors, Abl tyrosine kinase inhibitors, JAK tyrosine kinase inhibitors, TNF alpha ligand inhibitors, Hemoglobin modulators, TNF antagonists, proteasome inhibitors, CD3 modulators, Hsp 70 family inhibitors, Immunoglobulin agonists, CD30 antagonists, tubulin antagonists, Sphingosine-1-phosphate receptor-1 agonists, connective tissue growth factor ligand inhibitors, caspase inhibitors, adrenocorticotrophic hormone ligands, Btk tyrosine kinase inhibitors, Complement C1s subcomponent inhibitors, Erythropoietin receptor agonists, B-lymphocyte stimulator ligand inhibitors, Cyclin-dependent kinase-2 inhibitors, P-selectin glycoprotein ligand-1 stimulators, mTOR inhibitors, Elongation factor 2 inhibitors, Cell adhesion molecule inhibitors, Factor XIII agonists, Calcineurin inhibitors, Immunoglobulin G1 agonists, Inosine monophosphate dehydrogenase inhibitors, Complement C1s subcomponent inhibitors, Thymidine kinase modulators, Cytotoxic T-lymphocyte protein-4 modulators, Angiotensin II receptor antagonists, Angiotensin II receptor modulators, TNF superfamily receptor 12A antagonists, CD52 antagonists, Adenosine deaminase inhibitors, T-cell differentiation antigen CD6 inhibitors, FGF-7 ligands, dihydroorotate dehydrogenase inhibitors, CCR5 chemokine antagonists, CCR2 chemokine antagonists, Syk tyrosine kinase inhibitors, Interferon type I receptor antagonists, Interferon alpha ligand inhibitors, Macrophage migration inhibitory factor inhibitors, Integrin alpha-V/beta-6 antagonists, Cysteine protease stimulators, p38 MAP kinase inhibitors, TP53 gene inhibitors, Shiga like toxin I inhibitors, Fucosyltransferase 6 stimulators, Interleukin 22 ligands, CXCR1 chemokine antagonists, CXCR4 chemokine antagonists, IRS1 gene inhibitors, Protein kinase C stimulators, Protein kinase C alpha inhibitors, CD74 antagonists, Immunoglobulin gamma Fc receptor IIB antagonists, T-cell antigen CD7 inhibitors, CD95 antagonists, N acetylmannosamine kinase stimulators, Cardiotrophin-1 ligands, Leukocyte elastase inhibitors, CD40 ligand receptor antagonists, CD40 ligand modulators, IL-17 antagonists, TLR-2 antagonists, complement factor D inhibitors, complement factor B inhibitors, complement C5 inhibitors, MASP-2 inhibitors, MASP-3 inhibitors, C3 inhibitors, pegylated APL-1, C1s inhibitors, C6 inhibitors, and T cell receptor antagonists.

In some embodiments, the one or more additional therapeutic agents is selected from the group consisting of obinutuzumab, rituximab, ocrelizumab, cyclophosphamide, prednisone, hydrocortisone, hydrocortisone acetate, cortisone acetate, tixocortol pivalate, prednisolone, methylprednisolone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, halometasone, alclometasone dipropionate, beclomethasone, betamethasone valerate, betamethasone dipropionate, prednicarbate, clobetasone-17-butyrate, clobetasol-17-propionate, fluocortolone caproate, fluocortolone pivalate, fluprednidene acetate, hydrocortisone-17-butyrate, hydrocortisone-17-aceponate, hydrocortisone-17-buteprate, ciclesonide and prednicarbate, GB-0998, immuglo, begelomab, alefacept, aldesleukin, gevokizumab, daclizumab, basiliximab, inolimomab, beperminogene perplasmid, sirukumab, tocilizumab, clazakizumab, mepolizumab, fingolimod, panobinostat, triciribine, nilotinib, imatinib, tofacitinib, momelotinib, peficitinib, itacitinib, infliximab, PEG-bHb-CO, etanercept, ixazomib, bortezomib, muromonab, otelixizumab, gusperimus, brentuximab vedotin, Ponesimod, KRP-203, FG-3019, emricasan, corticotropin, ibrutinib, cinryze, conestat, methoxy polyethylene glycol-epoetin beta, belimumab, blisibimod, atacicept, seliciclib, neihulizumab, everolimus, sirolimus, denileukin diftitox, LMB-2, natalizumab, catridecacog, ciclosporin, tacrolimus, voclosporin, voclosporin, canakinumab, mycophenolate, mizoribine, CE-1145, TK-DLI, abatacept, belatacept, olmesartan medoxomil, sparsentan, TXA-127, BIIB-023, alemtuzumab, pentostatin, itolizumab, palifermin, leflunomide, PRO-140, cenicriviroc, fostamatinib, anifrolumab, sifalimumab, BAX-069, BG-00011, losmapimod, QPI-1002, ShigamAbs, TZ-101, F-652, reparixin, ladarixin, PTX-9908, aganirsen, APH-703, sotrastaurin, sotrastaurin, milatuzumab, SM-101, T-Guard, APG-101, DEX-M74, cardiotrophin-1, tiprelestat, ASKP-1240, BMS-986004, HPH-116, KD-025, OPN-305, TOL-101, defibrotide, pomalidomide, Thymoglobulin, laquinimod, remestemcel-L, Equine antithymocyte immunoglobulin, Stempeucel, LIV-Gamma, Octagam 10%, t2c-001, 99mTc-sestamibi, Clairyg, Prosorba, pomalidomide, laquinimod, teplizumab, FCRx, solnatide, foralumab, ATIR-101, BPX-501, ACP-01, ALLO-ASC-DFU, irbesartan+propagermanium, ApoCell, cannabidiol, RGI-2001, saratin, anti-CD3 bivalent antibody-diphtheria toxin conjugate, OMS-721, eculizumab, coversin, ACH-4471, ALN-CC5, AMY-101, IFX-1, IFX-2, IFX-3, LFG316, berinert, CB 2782, ANX005, APL-2, APL-1, PEG-Cp40, ALXN1007, bikaciomab, NOX-D20, NOX-D19, OMS906, mubodina, ALXN1210, ruconest, TNT009, SOBI005, SHP623, cinryze, lampalizumab, regenemab, RA101495, RA101295, zimura, NOX-100, LT-1951, and CD4+CD25+ regulatory T-cells.

F. Kits and Packages

The terms “kit” and “pharmaceutical kit” refer to a commercial kit or package comprising, in one or more suitable containers, one or more pharmaceutical compositions and instructions for their use. In one embodiment, kits comprising a compound of Formula (I), compound 1, or a subembodiment described herein, or a pharmaceutically acceptable salt thereof, and instructions for its administration are provided. In one embodiment, kits comprising a compound of Formula (I), compound 1, or a subembodiment described herein, or a pharmaceutically acceptable salt thereof, in combination with one or more (e.g., one, two, three, one or two, or one to three) additional therapeutic agents and instructions for their administration are provided.

In one embodiment, the compounds of this disclosure are formulated into administration units which are packaged in a single packaging. The single packaging encompasses but is not limited to a bottle, a child-resistant bottle, an ampoule, and a tube. In one embodiment, the compounds of this disclosure and optionally additional therapeutic agents, are formulated into administration units and every single administration unit is individually packaged in a single packaging. Such individually packaged units may contain the pharmaceutical composition in any form including but not limited to liquid form, solid form, powder form, granulate form, an effervescent powder or tablet, hard or soft capsules, emulsions, suspensions, syrup, suppositories, tablet, troches, lozenges, solution, buccal patch, thin film, oral gel, chewable tablet, chewing gum, and single-use syringes. Such individually packaged units may be combined in a package made of one or more of paper, cardboard, paperboard, metal foil and plastic foil, for example a blister pack. One or more administration units may be administered once or several times a day. One or more administration units may be administered three times a day. One or more administration units may be administered twice a day. One or more administration units may be administered on a first day and one or more administration units may be administered on the following days.

### EXAMPLES

### Example 1: Study of Compound 1 in a Patient with Progressive Complement 3 Glomerulonephritis

Under the Special Needs program in the United Kingdom (which is similar to a compassionate use protocol in the US), a C3 glomerulonephritis patient received treatment with the orally administered complement inhibitor compound 1, following the protocol detailed below. The patient had refractory disease despite a kidney transplant and prior treatment with the broadly immunosuppressive drugs rituximab, cyclophosphamide, mycophenolate mofetil, tacrolimus, and steroids. Renal allograft biopsies were taken pre-dose, 2 and 7 months during therapy.

### Results:

The patient's condition improved in response to compound 1 treatment. The improvement seen with compound 1 treatment in this patient was based on the on-treatment kidney biopsy histologic findings that showed clearance of glomerular endocapillary proliferation and a marked decrease in glomerular inflammatory macrophages compared to the pre-treatment biopsy. Proteinuria dropped approximately 80% with compound 1 treatment.

Estimated glomerular filtration rate (eGFR) was 83 mL/min/1.73 m² 14 months prior to treatment with compound 1 and deteriorated to 46 mL/min/1.73 m² when treatment with compound 1 was started. Treatment with compound 1 attenuated or stopped the eGFR decline.

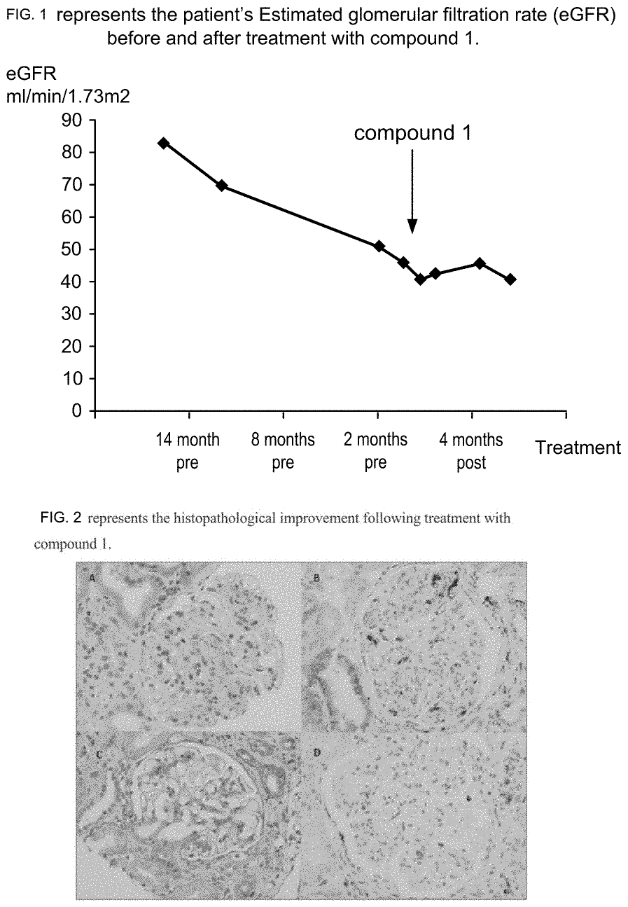

After 1 month of treatment, the eGFR decline was already attenuated (FIG. 1 shows the eGFR prior and after treatment with compound 1). Repeat biopsies showed resolution of glomerular endocapillary hypercellularity and reduction in glomerular macrophages. Compound 1 stabilized eGFR and reduced glomerular inflammation.

TABLE 1
Endocapillary hypercellularity, Immunoflourescence
microscopy and CD68-positive cells/
glomerulus at different time points.

| | Endocapillary | Immuno- | CD68-positive |
|---|---|---|---|
| | hypercellularity/ | flourescence | cells/ |
| | total | microscopy | glomerulus |
| Pre-treatment | 3/11 | C3, 2+; IgM, trace | 11 |
| 2 months | 0/36 | C3, 2+; IgM, neg | 2-3 |
| 7 months | 0/14 | C3, 2+; IgM, neg | 1-2 |

FIG. 2 represents the histopathological improvement following treatment with compound 1.

(A) Haemotoxylin and Eosin (H&E) staining before treatment with compound 1 demonstrates fibrinoid necrosis and multiple inflammatory cells.
(C) Periodic acid-Schiff (PAS) staining after treatment with compound 1 shows a reduction in endocapillary hypercellularity and glomerular inflammation.
(B) CD68 staining before treatment with compound 1.
(D) CD68 staining after treatment with compound 1 demonstrates a reduction in glomerular macrophages.

### Protocol of the Study:

### Aim

The aim of this study is to evaluate the efficacy, safety, and tolerability of compound 1 in a patient with progressive complement 3 (C3) glomerulonephritis.

### Objectives

The primary safety objective of this study is to evaluate the safety and tolerability of compound 1.

The primary efficacy objective is to evaluate the efficacy of compound 1 based on change from baseline in eGFR (MDRD, Estimated Glomerular Filtration Rate) and proteinuria.

The secondary objectives of this study include assessment of:
1. Change from baseline in pharmacodynamic markers in plasma and urine, e.g., MCP-1, C3a, C5a, properdin, and sC5b-9;
2. Change from baseline in glomerular pathology based on renal biopsy;
3. Evaluation of the plasma concentrations of compound lin C3 glomerulonephritis.

### Methodology

This is a clinical study to test the safety, tolerability, and efficacy of compound 1 in a patient with recurrent C3GN in a renal transplant.

The patient will have biopsy proven recurrent C3GN prior to start of dosing, and be deemed eligible based on the inclusion and exclusion criteria. Screening procedures will include recording of demographics, medical history, medication history, physical examination and vital signs, serum chemistry, hematology, urinalysis (including UPCR measurement), viral screening (if not performed within prior 12 weeks), and estimated glomerular filtration rate (eGFR) assessment based on serum creatinine. The baseline eGFR needs to be at least 25 mL/min/1.73 m² for study eligibility.

On Day 1, the patient will start compound 1 treatment. Patients will take compound 1 30 mg orally twice daily for an initial period of 84 days. The patient will visit the study center on Days 1, 8, 15, 29, 57, and 85. The compound 1 dose will be taken in the morning optimally within one hour after breakfast and in the evening optimally within one hour after dinner. If the patient's clinical condition stabilizes or improves, and there are no adverse events preventing further treatment, the patient may be treated for another 84-day treatment cycle. The 84-day cycles may be repeated for a total of up to 4 cycles under this protocol. For the 84-day cycles after the first cycle, the patient will visit the study center every 4 weeks. There will be 4-week follow-up period after the patient stops the compound 1 treatment.

At the Day 1 and post-Day 1 study visits, blood and urine samples will be collected for safety, efficacy, and pharmacokinetic measurements. Physical examinations and vital signs assessments will be performed throughout the study. Concomitant medication and adverse event assessments will be made at every study visit. If at all possible, a renal biopsy will be performed after an appropriate follow-up period to assess the changes in kidney histology.

No new treatment for C3GN may be added during the study period (active treatment period or follow up), unless the subject's condition deteriorates to the extent that the investigator deems it in the best interest of the subject to do so.

Duration of treatment with compound 1: 84 days with up to 3 repeats of the 84-day cycle for a total period of up to 336 days.

Duration of follow up after end of treatment with study medicine: 4 weeks.

The patient's condition will be evaluated by the Investigator at the end of the study and appropriate standard of care medical treatment will be provided as needed.

Main Criteria for Inclusion
1. Biopsy-proven C3 GN based on a renal biopsy within 8 weeks prior to screening;
2. eGFR≥25 mL/min/1.73 m² (by MDRD equation);
3. If having a partner of childbearing potential, must use adequate contraception throughout the study and for at least 3 months after completion of dosing; Adequate contraception is defined as resulting in a failure rate of less than 1% per year (combined estrogen and progestogen [oral, intravaginal, or transdermal], or progestogen-only hormonal contraception (oral, injectable, or implantable), intra-uterine device, intra-uterine hormone releasing system, bilateral tubal occlusion, vasectomized partner, or sexual abstinence);
4. Willing and able to give written Informed Consent and to comply with the requirements of the study protocol; and
5. Judged to be otherwise healthy by the Investigator, based on medical history, physical examination, and clinical laboratory assessments. Clinical laboratory values that are outside of normal limits (other than those specified in the Exclusion Criteria) and/or with other abnormal clinical findings that are judged by the Investigator not to be of clinical significance, may be allowed.

Main Criteria for Exclusion
1. Proteinuria>8 g/day (or >8 g/g creatinine);
2. Use of eculizumab within 26 weeks prior to dosing;
3. History or presence of any form of cancer within the 5 years prior to screening, with the exception of excised basal cell or squamous cell carcinoma of the skin, or carcinoma in situ such as cervical or breast carcinoma in situ that has been excised or resected completely and is without evidence of local recurrence or metastasis;
4. Positive HBV, HCV, or HIV viral screening test;
5. Any infection requiring antibiotic treatment that has not cleared prior to starting compound 1 treatment on Day 1;
6. WBC count less than 4000/μL, or neutrophil count less than 2000/μL, or lymphocyte count less than 1000/μL;
7. Hemoglobin less than 9 g/dL (or 5.56 mmol/L) at screening;
8. Evidence of hepatic disease; AST, ALT, alkaline phosphatase, or bilirubin>3× the upper limit of normal;
9. Participated in any clinical study of an investigational product within 30 days prior to screening or within 5 half-lives after taking the last dose; and
10. History or presence of any medical condition or disease which, in the opinion of the Investigator, may place the subject at unacceptable risk for study participation.

### Duration of Treatment and Observation

The patient will be screened within a period not to exceed 21 days prior to Day 1. The compound 1 treatment period is at least 84 days and up to 336 days, and the patient will be followed for 4 weeks (28 days) after dosing is stopped.

To the extent possible, any adverse events that are deemed study drug-related and are ongoing at discharge will be followed-up to resolution or until a determination is made that the unresolved event is stable. The patient's condition will be evaluated by the Investigator at the end of the study and appropriate standard of care medical treatment will be provided as needed.

### Safety Assessments

Safety assessments include adverse events, physical examination abnormalities, vital signs, and clinical laboratory tests (including blood chemistry, hematology, and urinalysis).

### Efficacy Assessments

Efficacy assessments include:
1. First morning urinary PCR or ACR;
2. eGFR by Modification of Diet in Renal Disease (MDRD) formula based on serum creatinine;
3. Plasma and urine pharmacodynamic markers, e.g., MCP-1, C3a, C5a, properdin, and sC5b-9;
4. Glomerular inflammation (e.g., crescents, inflammatory cell infiltrate, endocapillary proliferation) and C3 deposition in a follow-up renal biopsy sample;

### Pharmacokinetic Assessments

Concentrations of compound 1 and possible metabolites will be determined in plasma from 2-mL blood samples collected in EDTA tubes on Days 8, 15, 29, 57, and 85. The date and time of the last dose of compound 1 prior to sample collection for compound 1 measurement will be recorded. The samples will be kept frozen at −70° C. or lower and shipped on dry ice for assay.

Plasma samples will continue to be collected every 4 weeks during any subsequent 84-day cycles.

### Pharmacodynamic Markers

Plasma samples will be collected on Day 1 (pre-dose), and Days 8, 15, 29, 57, and 85 for pharmacodynamic marker measurements, including, for example, complement fragments, and inflammatory cytokine and chemokine levels. Urine samples will also be collected on Day 1 (pre-dose) and Days 8, 15, 29, 57, and 85 for biomarker assessments including, for example, MCP-1, complement fragments, and inflammatory chemokine and cytokine levels.

Plasma and urine samples will continue to be collected every 4 weeks during any subsequent 84-day cycles.

### Renal Histology

Renal biopsies will be analyzed by periodic acid-Schiff (PAS) staining, immunofluorescence staining for C3, C5b-9, and potentially other markers. Electron microscopy may also be performed.

### Statistical Methods

### Demographics and Baseline Characteristics

All patient baseline characteristics and demographic data (age, sex, race, ethnicity, weight, height, body mass index, smoking status, viral test results, C3 GN disease duration (from time of first diagnosis based on renal biopsy), renal transplant history, eGFR, proteinuria (PCR), urinary MCP-1:creatinine ratio, physical examination abnormalities, medical history, previous (within 6 months of screening) and concomitant medications (including other treatments for C3 GN) at study entry will be listed.

### Safety Analysis

The primary safety endpoint is the patient incidence of adverse events.

Other safety endpoints include:
1. Change from baseline in all safety laboratory parameters;
2. Change from baseline in vital signs.

All clinical safety and tolerability data will be listed. Treatment-emergent adverse events will be listed by System Organ Class, by relatedness and by maximum severity. Serious adverse events and adverse events leading to withdrawal will be listed. Vital signs and change from baseline in vital signs will be listed by study visit. Laboratory data (actual values and change from baseline) will be listed by study visit. Abnormal laboratory values will be flagged.

### Efficacy Analysis

The primary efficacy endpoints are the change from baseline over the treatment period in eGFR and first morning urinary PCR.

Other efficacy endpoints include:
1. The percent change from baseline in plasma and urinary biomarkers, e.g., MCP-1, C3a, C5a, properdin, and sC5b-9;
2. Change from baseline to follow-up biopsy in glomerular inflammation (crescents, inflammatory cell infiltrate, and endocapillary proliferation), C3 deposits, and C5b-9 deposits.

Change and percent change in the efficacy parameters during the 4-week follow-up period will also be assessed to determine the off-treatment effect.

### Pharmacokinetic Analysis

Plasma samples will be collected on Days 8, 15, 29, 57, and 85 to determine the plasma concentrations of compound 1 (and metabolites). Plasma concentrations of compound 1 will be listed and plotted by study visit.

### Example 2. A Randomized, Double-Blind, Placebo-Controlled Phase 2 Study to Evaluate the Safety and Efficacy of Compound 1 in Patients with C3 Glomerulopathy

### Protocol of the Study Planned

### Aim

The aim of this study is to evaluate the effect of compound 1 treatment on renal disease activity in patients with complement 3 glomerulopathy (C3G). The intent is to slow down or improve renal disease with compound 1 treatment in these patients.

### Objectives

The primary objective is to evaluate the efficacy of compound 1 compared to placebo based on histologic changes in C3G pathology from kidney biopsies taken before and during treatment.

The secondary objectives of this study include assessment of:
1. The safety of compound 1 compared to placebo based on the incidence of adverse events, changes in clinical laboratory measurements, and vital signs;
2. Changes in laboratory parameters of renal disease including estimated glomerular filtration rate (eGFR), proteinuria, and urinary excretion of monocyte chemoattractant protein-1 (MCP-1) with compound 1 compared to placebo;
3. Health-related quality-of-life changes based on Short Form-36 version 2 (SF-36 v2) and EuroQOL-5D-5L (EQ-5D-5L) with compound 1 compared to placebo;
4. Evaluation of the pharmacokinetic profile of compound 1 in patients with C3 glomerulopathy.

Additionally, changes from baseline in markers of alternative complement pathway involvement, e.g., C3, C3d, C3c, C3adesArg, C5, C5a, C5b-9, C5adesArg, and other markers of inflammation, may be assessed in plasma/serum or urine over the course of the treatment period.

### Methodology

This is a Phase 2 study to test the efficacy, safety, and tolerability of compound 1 in patients with C3G, including both C3GN and DDD. Eligible patients will be stratified based on two factors:
1. C3GN or DDD, and
2. Having received a kidney transplant or not, prior to randomization.

Patients will then be randomized, 1:1, to receive 30 mg compound 1 twice daily or matching placebo for 26 weeks in a double-blind, placebo-controlled manner. The 26-week double-blind period will be followed by a 26-week period during which all patients will receive compound 1 treatment.

Patients will be screened for enrollment based on biopsy proven C3 glomerulopathy (i.e., ≥2-levels of magnitude greater staining of C3 than any combination of IgG, IgM, IgA, and Clq) and evidence of inflammation based on leukocyte infiltration and/or endocapillary proliferation.

The screening period will be up to 28 days. Screening procedures will include written informed consent, demographics, medical history, medication history, physical examination and vital signs, 12-lead ECG, serum pregnancy test for women of childbearing potential, serum chemistry (including serum creatinine), hematology, urinalysis, urinary protein:creatinine ratio (PCR), viral and TB screening. If a patient did not have a renal biopsy in the past 12 weeks, a renal biopsy needs to be done prior to dosing. Prior to starting study drug treatment, blood samples will be collected for the following measurements to create a baseline profile for all patients:
1. C3, C3d, C3c, C3adesArg, and C4;
2. C3 nephritic factor;
3. C5, C5a, C5b-9, C5adesArg;
4. Serum complement Factor H and factor B;
5. Serum paraprotein detection;
6. Complement factor H related protein 5 (CFHR5) mutation.

Patients meeting inclusion criteria will start study drug treatment on Day 1. Patients will take compound 1 30 mg or matching placebo orally twice daily. The treatment period is 52 weeks (364 days). The study drug will be taken in the morning preferably with food and in the evening preferably with food, approximately 12 hours after the morning dose. Patients who receive placebo during the first 26 weeks, will receive compound 1 in a blinded cross-over. After the 364-day treatment period, all patients will be followed for 8 weeks (56 days) without study drug treatment.

At post-Day 1 study visits, blood and urine samples will be collected for safety, efficacy, and pharmacokinetic and biomarker measurements. A serum pregnancy test for women of childbearing potential will be done regularly during the 52-week treatment period and at the end of the 8-week follow-up period. Physical examinations and vital signs assessments will be performed throughout the study. Health-related quality of life using the EQ-5D-5L and SF-36 v2 surveys will be assessed periodically over the course of the study. Study drug will be dispensed and drug accountability will be done. Concomitant medication and adverse event assessments will be made at every study visit. A follow-up renal biopsy will be performed at the following time points:
1. After the 26-week placebo-controlled treatment period;
2. If a patient is withdrawn early from the study, and
3. After the 52-week treatment period.

If a patient is on other immunosuppressive treatment at the start of dosing, the dose(s) of concomitant immunosuppressive treatment may not be increased during the study. Treatment with these other drugs may be reduced or stopped during the study, if the patient's condition justifies it. No new treatments may be added during the study period (active treatment period or follow up), unless the patient's condition deteriorates to the extent that the investigator deems it in the best interest of the patient to do so. This will be considered a treatment failure.

Patients who experience deteriorating renal function based on an increase in serum creatinine of at least 50% (confirmed by a repeat measurement after 2 weeks) which is otherwise not explained (e.g., dehydration, new medication), or an increase in proteinuria of >3 g/g creatinine from baseline or to a level>8 g/g (confirmed by a repeat measurement after 2 weeks) during the 52-week treatment period, will exit the treatment phase of the study and be treated at the discretion of their doctor. They will remain in the study for follow up and outcome recording. These will be considered treatment failures.

For study centers where enrollment of adolescents (12 to 17 years old) is approved, compound 1 or placebo dosing will initially be given based on the body weight at screening and the dose will be adjusted based on compound 1 plasma levels as shown in the table below.

Only in 12 to 17 year old patients, blood samples will be taken pre-dosing and at Hours 0.5, 1, 2, 3, 4, and 6 after the first compound 1 dose on Day 1 and plasma samples will be sent to the central laboratory for expeditious measurement of compound 1 and its metabolite in these patients. Dose adjustments will be made based on AUC₀₋₆ as shown in the table below. These AUC₀₋₆ thresholds are based on the mean compound 1 plasma exposure (525 ng·hr/mL) and one standard deviation (174 ng·hr/mL) above or below the mean in adult patients from Phase 2 study CL002_168 in AAV.

| | | compound 1 | |
|---|---|---|---|
| | Initial | Plasma | |
| | compound | AUC₀₋₆ | compound 1 |
| | 1/placebo | (ng · hr/mL) | Dose |
| Body weight | dose | on Day 1 | Adjustment |
| 40 kg (88 lb) | 10 mg | ≥351 | None |
| | twice daily | | |
| | | <351 | Increase dose to |
| | | | 20 mg twice daily |
| 40-55 kg (88-121 lb) | 20 mg | 351 to 699 | None |
| | twice daily | | |
| | | <351 | Increase dose to |
| | | | 30 mg twice daily |
| | | >699 | Decrease dose to |
| | | | 10 mg twice daily |
| >55 kg (121 lb) | 30 mg | ≤699 | None |
| | twice daily | | |
| | | >699 | Decrease dose to |
| | | | 20 mg twice daily |

Patients will visit the study center during Screening and on Day 1 (baseline) and Weeks 1, 2, 4, 8, 12, 16, 20, 26, 32, 38, 44, 52, and 60.

Duration of double-blind treatment with compound 1 or placebo: 26 weeks.

Duration of treatment with compound 1 after the double-blind treatment period: 26 weeks.

Duration of follow up after end of treatment with study medicine: 8 weeks.

Patients will be discharged from the study when all the Week 60 visit procedures have been completed. The patient's condition will be evaluated by the Investigator at the end of the clinical trial (Week 60) and appropriate standard of care medical treatment will be provided to all patients as needed.

### Number of Patients

Approximately 44 male or female patients with C3 glomerulopathy will be enrolled in this study. Patients who drop out before the Week 26 visit may be replaced.

Main Criteria for Inclusion
1. Biopsy-proven C3 glomerulopathy, either DDD or C3GN, with 2-levels of magnitude greater staining of C3 than any combination of IgG, IgM, IgA, and C1q, and with evidence of inflammation, based on leukocyte infiltration or endocapillary proliferation, observed in a renal biopsy taken within 12 weeks prior to screening or during screening; patients with a kidney transplant are eligible for the study;
2. Plasma C5b-9 above the upper limit of the reference range of the central laboratory;
3. Male or female patients, aged at least 18 years; where approved, adolescents (12-17 year old) may be enrolled; female patients of childbearing potential may participate if adequate contraception is used during, and for at least the three months after study completion; Male patients with partners of childbearing potential may participate in the study if they had a vasectomy at least 6 months prior to randomization or if adequate contraception is used during, and for at least the three months after study completion; Adequate contraception is defined as resulting in a failure rate of less than 1% per year (combined estrogen and progestogen [oral, intravaginal, or transdermal], or progestogen-only hormonal contraception (oral, injectable, or implantable), intra-uterine device, intra-uterine hormone releasing system, bilateral tubal occlusion, vasectomized partner, or sexual abstinence);
4. Willing and able to give written Informed Consent and to comply with the requirements of the study protocol; written Informed Consent should be obtained from the legal guardian in accordance with regional laws or regulations for patients 12 to 17 years of age; and
5. Judged to be otherwise fit for the study by the Investigator, based on medical history, physical examination, and clinical laboratory assessments. Patients with clinical laboratory values that are outside of normal limits (other than those specified in the Exclusion Criteria) and/or with other abnormal clinical findings that are judged by the Investigator not to be of clinical significance, may be entered into the study.

Main Criteria for Exclusion
1. Pregnant or nursing;
2. Proteinuria>8 g/day (or >8 g/g creatinine);
3. More than 50% interstitial fibrosis on renal histology;
4. Use of eculizumab within 26 weeks prior to dosing;
5. Secondary C3 disease, e.g., infection-associated disease, or associated with another systemic or autoimmune disease;
6. Currently on dialysis or likely will require dialysis within 7 days;
7. History or presence of any form of cancer within the 5 years prior to screening, with the exception of excised basal cell or squamous cell carcinoma of the skin, or carcinoma in situ such as cervical or breast carcinoma in situ that has been excised or resected completely and is without evidence of local recurrence or metastasis;
8. Positive HBV, HCV, or HIV viral screening test;
9. Evidence of tuberculosis based on interferon γ release assay (IGRA), tuberculin purified protein derivative (PPD) skin test, or chest radiography done at screening or within 6 weeks prior to screening;
10. WBC count less than 3500/uL, or neutrophil count less than 1500/uL, or lymphocyte count less than 800/uL before start of dosing;
11. Evidence of hepatic disease; AST, ALT, alkaline phosphatase, or bilirubin>3× the upper limit of normal before start of dosing;
12. Known hypersensitivity to compound 1 or inactive ingredients;
13. Participated in any clinical study of an investigational product within 30 days prior to screening or within 5 half-lives after taking the last dose; and
14. History or presence of any medical condition or disease which, in the opinion of the Investigator, may place the patient at unacceptable risk for study participation.

### Duration of Treatment and Observation

Patients will be screened within a period not to exceed 28 days prior to Day 1. The treatment period is 52 weeks (364 days) and all patients will be followed for 8 weeks (56 days) after the dosing period.

To the extent possible, any adverse events that are deemed study drug-related and are ongoing at discharge will be followed-up to resolution or until a determination is made that the unresolved event is stable. The patient's condition will be evaluated by the Investigator at the end of the clinical trial and appropriate standard of care medical treatment will be provided to all patients as needed.

### Safety Assessments

Safety assessments include adverse events, physical examination abnormalities, vital signs, and clinical laboratory tests (including blood chemistry, hematology, and urinalysis).

### Efficacy Assessments

Efficacy assessments include:
1. Renal histology to determine the C3G Histologic Index (CHI) for disease activity and chronicity;
2. eGFR calculated by the Modification of Diet in Renal Disease (MDRD) equation from serum creatinine;
3. First morning urinary PCR;
4. First morning urinary MCP-1:creatinine ratio;
5. EQ-5D-5L and SF-36 v2.

### Pharmacokinetic Assessments

Concentrations of compound 1 and metabolites will be determined in plasma according to the Time and Events Table.

### Pharmacodynamic Markers

Plasma/serum samples will be collected according to the Time and Events Table for pharmacodynamic marker measurements, including, for example, complement fragments, and inflammatory cytokine and chemokine levels. Urine samples will also be collected according to the Time and Events Table for biomarker assessments including, for example, complement fragments, sCD163, and inflammatory chemokine and cytokine levels.

### Renal Histology

For eligibility assessment, renal biopsy samples will be assessed by immunofluorescence staining for C3 and immunoglobulins. Patients must have biopsy-proven C3 glomerulopathy, either DDD or C3GN, with ≥2-levels of magnitude greater staining of C3 than any combination of IgG, IgM, IgA, and Clq, and with evidence of inflammation, based on leukocyte infiltration or endocapillary proliferation, observed in a renal biopsy taken within 12 weeks prior to screening or during screening.

All renal biopsies will also be analyzed based on hematoxylin-eosin (H&E) staining, periodic acid-Schiff (PAS) staining, trichrome, and Jones methenamine silver staining. These renal biopsies will be evaluated by a central reader, blinded to treatment assignment from either slides or high-resolution electronic images.

The central reader will determine the degree of disease activity and chronicity.

### Statistical Methods

### Demographics and Baseline Characteristics

All patient baseline characteristics and demographic data (age, sex, race, ethnicity, weight, height, body mass index, viral test results, C3 glomerulopathy disease duration (from time of first diagnosis based on renal biopsy), eGFR, proteinuria (PCR), complement marker levels, urinary MCP-1:creatinine ratio, physical examination abnormalities, medical history, previous (within 6 months of screening) and concomitant medications (including other treatments for C3 glomerulopathy) at study entry will be listed by study center and patient number, and will also be summarized.

### Efficacy Analysis

The primary efficacy endpoint is the percent change from baseline to week 26 in the C3G Histologic Index (CHI) for disease activity. The compound 1 and placebo groups will be compared by ANCOVA with treatment group and randomization strata (C3GN or DDD, and renal transplant or not) as factors, and baseline as covariate. Point estimates and corresponding 95% confidence intervals will be estimated for the difference between the compound 1 and placebo control group.

Since the placebo group will receive compound 1 during the second 26 weeks of the study, the change from Week 26 to Week 52 in the CHI in the placebo control group will be compared to the change from baseline to Week 26 in this group. This analysis will be done by the paired t-test. Point estimates and corresponding 95% confidence intervals will be estimated for the difference between the second 26 weeks (compound 1 treatment) and the first 26 weeks (placebo treatment).

The change from baseline to Week 52 in the CHI will also be compared to the change from baseline with Week 26 in placebo control group using similar methodology as described for the primary efficacy endpoint.

Other efficacy endpoints include:
1. The percent change from baseline in the CHI for disease chronicity over the placebo-controlled 26-week treatment period;
2. The change and percent change from baseline in eGFR over the placebo-controlled 26-week treatment period;
3. The percent change from baseline in urinary PCR over the placebo-controlled 26-week treatment period;
4. The percent change from baseline in urinary MCP-1:creatinine ratio over the placebo-controlled 26-week treatment period;
5. Change from baseline in EQ-5D-5L and SF-36 v2 (domains and component scores) over the placebo-controlled 26-week treatment period.

Continuous variables, including eGFR, urinary PCR, urinary MCP-1:creatinine ratio, EQ-5D-5L, and SF-36 v2 will be analyzed using a mixed effects model for repeated measures (NMMR) with treatment group, visit, treatment-by-visit interaction, and randomization strata (C3GN or DDD, and renal transplant or not) as factors, and baseline as covariate. Patients will be considered as repeated measure units over visits. Point estimates and corresponding 95% confidence intervals will be estimated for the difference between the compound 1 group and the control group across 26 weeks using simple contrast from the model. Similar to the primary endpoint, the second 26 weeks will be compared to the first 26 weeks for the placebo group.

Change and percent change in the efficacy parameters during the 8-week follow-up period will also be assessed to determine the off-treatment effect.

Change from baseline in markers of alternative complement pathway activation will be reported.

Summary statistics will be calculated for each of the efficacy endpoints. For continuous variables, numbers, means, medians, ranges, standard deviations, standard errors, and 95% confidence intervals will be calculated. Geometric means will be calculated for urinary PCR and MCP-1:creatinine, and other measurements that are not normally distributed.

### Safety Analysis

Safety endpoints include:
1. Patient incidence of treatment-emergent serious adverse events, adverse events, and withdrawals due to adverse events;
2. Change from baseline and shifts from baseline in all safety laboratory parameters;
3. Change from baseline in vital signs.

All patients who are randomized and received at least one dose of study medication will be included in the safety population.

All clinical safety and tolerability data will be listed by treatment group and by patient, and will be summarized by treatment group.

All reported adverse events will be coded using MedDRA and listed by System Organ Class, preferred term, and verbatim term.

Treatment-emergent adverse events will be listed and summarized by treatment group by System Organ Class, by relatedness and by maximum severity.

Treatment-emergent serious adverse events and adverse events leading to withdrawal will be summarized by treatment group.

Individual vital signs and change from baseline in vital signs will be listed by treatment group, patient, and study visit, and summarized by treatment group.

Laboratory data (actual values and change from baseline) will be listed by treatment group, patient, and study visit. Abnormal laboratory values will be flagged. Laboratory data will also be summarized by treatment group and study visit. Shift tables will be generated for shifts in laboratory parameters by study visit.

### Pharmacokinetic and Pharmacodynamic Marker Analysis

Plasma samples will be collected over the course of the study to determine the PK profile of compound 1 (and metabolites). Individual plasma concentrations of compound 1 (and metabolites) will be listed, plotted, and summarized descriptively and graphically. PK parameters will be calculated based on plasma compound 1 concentrations at the time of sample collection in relation to time of administration of the most recent dose of study medication. PK parameters of significant metabolites may also be calculated.

Plasma and urinary PD markers will be summarized and may be analyzed using methods analogous to the efficacy parameters. The following parameters will be determined, where possible, in 12-17 year old patients:

Cmax Maximum plasma concentration
tmax Time of maximum plasma concentration
AUC₀₋₆ Area under the plasma concentration-time curve from Time 0 to Hour 6 on Day 1
Cmin Trough level plasma concentrations at post-Day 1 visits

The relationship between PK parameters and renal function based on eGFR will be evaluated. The data may also be used to evaluate the PK/PD relationship of compound 1 treatment. To this end, the change and/or percent change from baseline in urinary PCR, eGFR, urinary MCP-1:creatinine ratio, and other biomarkers may be used as PD markers.

### Example 3. Summary of Top-Line Results from Phase II Trial

The primary endpoint of the study was change from baseline to week 26 in the C3G Histologic Index for Disease Activity, comparing the changes in kidney histology from biopsy sections taken from patients characterized by elevated levels of C5b-9 complement markers in the blood at time of study entry (baseline). Biopsies, taken at baseline and after 26 weeks of treatment showed that the placebo group worsened by 38% on average in the C3G Activity Score while the avacopan group improved by 2% on average. This approximately 40% average difference between the two treatment arms did not constitute statistical significance due to the high patient to patient variability. Comparison of the C3G Activity Score of all C3G subjects (comprising those with both elevated levels of C5b-9 as well as those with normal levels of C5b-9) yielded similar results: the placebo group worsened by 26% on average in the C3G Activity Score, while avacopan therapy resulted in an improvement of 6% on average.

Importantly, those patients who received avacopan experienced significant benefits in terms of kidney function and other parameters compared to those who received placebo. These benefits, assessed as pre-specified secondary endpoints, include: Slowing of Fibrosis Progression

Avacopan therapy demonstrated evidence for a significant slowing of the progression of fibrosis as assessed by the C3G Histologic Index for Disease Chronicity. The placebo group overall exhibited a 26 percentage point higher change from baseline to Week 26 in the C3G Index for Disease Chronicity than avacopan (58% versus 32%, respectively) representing a worsening in disease chronicity. The mean change from baseline to week 26 was 1.6 for placebo versus 0.8 for avacopan (P<0.05). The avacopan-related lower increase is notable because published literature shows that each 1-unit increase in C3G Histologic Index for Disease Chronicity from baseline increases by 59% (P<0.001) the risk of doubling of creatinine, progression to chronic kidney disease stage 5, ESRD requiring dialysis or transplantation, or death.¹

### Improvement in Kidney Function

The avacopan group demonstrated a statistically significant improvement in eGFR from baseline to week 26. Overall, the eGFR in the avacopan group improved 5% on average from baseline while the placebo group worsened by 6% (P=0.0221). Renal improvement was particularly pronounced for C3G subjects with eGFR of <60 mL/min/1.73 m² at baseline, as their eGFR on average increased relative to placebo by nearly 20% after 26 weeks (13% improvement for avacopan versus 6% worsening from baseline for placebo, P=0.0199). This was equivalent to a mean increase of about 5 mL/min/1.73 m² on avacopan versus a decrease of 1.4 mL/min/1.73 m² in the placebo group. Significant improvement in eGFR in a blinded comparative setting over 26 weeks has not been noted in C3G studies prior to this.

Other measures of kidney function include UPCR (proteinuria), where high UPCR is known to be associated with higher risk of ESRD in C3G as well as other renal diseases, and urinary MCP-1 creatinine ratio, a marker of glomerular inflammation.

In the ACCOLADE study, avacopan therapy was associated with a rapid reduction in UPCR (proteinuria). From baseline, a progressive proteinuria drop was seen in the avacopan group: at week 16 there was a 35% mean decrease in UPCR with avacopan versus a 1% decrease in the placebo group (P<0.05), and at the end of 26 weeks UPCR was reduced by 26% in the avacopan group versus 14% in the placebo group.

Similar reductions were seen in urinary MCP-1 creatinine ratio the avacopan group versus the placebo group throughout the 26-week treatment period, with the avacopan group consistently exhibiting lower levels of the kidney inflammation marker being shed in the urine.

| | Placebo | Avacopan |
|---|---|---|
| | Group | Group |
| Urinary MCP-1:Creatinine Ratio | 1% | −12% |
| Percent Change from Baseline | | |
| Urinary Protein:Creatinine Ratio | −14% | −26% |
| (Proteinuria) Percent Change from Baseline | | |

Avacopan also appeared safe and well-tolerated in patients with C3G.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of treating a human suffering from or susceptible to complement 3 glomerulopathy comprising administering to the human an effective amount of a compound having formula (I) 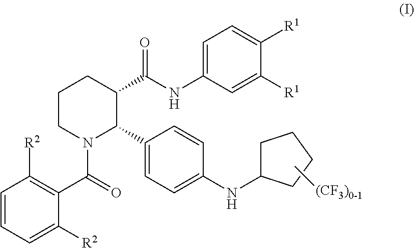
or a pharmaceutically acceptable salt thereof, wherein said therapeutically effective amount is about 10 mg or 30 mg of the compound twice daily, wherein
each R¹ is independently selected from the group consisting of CH₃, CF₃, CH₂CH₃, Cl, 1-pyrrolidine, ˜O—CH(CH₃)₂, and CH₂OH; and
each R² is independently selected from the group consisting of CH₃ and F.

2. The method of claim 1, wherein humans with an eGFR of <60 mL/min/1.73 m² at baseline have a significant improvement in renal function relative to placebo.

3. The method of claim 2, wherein the change in eGFR from baseline to after week 26 in humans receiving a compound of Formula I is at least a 10% improvement.

4. The method of claim 2, wherein the change in eGFR from baseline to after week 26 in humans receiving placebo is at least a 5% worsening.

5. The method of claim 1, wherein the human has a baseline C5b-9 blood plasma concentration of >244 ng/mL.

6. The method of claim 1, wherein the human has a baseline C5b-9 blood plasma concentration of ≤244 ng/mL.

7. The method of claim 1, wherein the human has a high baseline C3, C3d, C3c, C3adesArg, or C4 plasma level.

8. The method of claim 1, wherein the human has a low baseline C3, C3d, C3c, C3adesArg, or C4 plasma level.

9.-20. (canceled)

21. The method of claim 1, wherein the human has a complement protein plasma level baseline that is 20% or more below the average plasma level for the complement protein in healthy individuals who are not diagnosed with C3G.

22. The method of claim 21, wherein the complement protein is selected from the group consisting of C2, C3, C3d, C3c, C3adesArg, C4, C5a, C5b-9, and C5adesArg.

23. The method of claim 21, wherein the complement protein is C4.

24. The method of claim 1, wherein the compound is 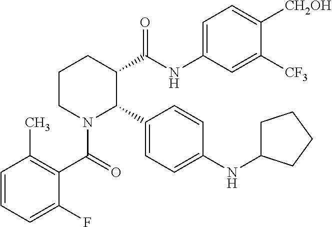 [SMILES: Cc1cccc(F)c1C(=O)N1CCC[C@H](C(=O)Nc2ccc(CO)c(C(F)(F)F)c2)[C@@H]1c1ccc(NC2CCCC2)cc1]
or a pharmaceutically acceptable salt thereof.

25. The method of claim 1, wherein the compound is 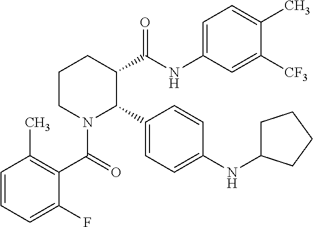 [SMILES: Cc1ccc(NC(=O)[C@H]2CCCN(C(=O)c3c(C)cccc3F)[C@H]2c2ccc(NC3CCCC3)cc2)cc1C(F)(F)F]
or a pharmaceutically acceptable salt thereof.

26. The method of claim 1, wherein the human suffers from a disease selected from the group consisting of complement 3 glomerulonephritis, progressive complement 3 glomerulonephritis, recurrent complement 3 glomerulonephritis after a renal transplant, recurrent complement 3 glomerulonephritis after a renal transplant, and dense deposit disease.

27.-29. (canceled)

30. The method of claim 1, wherein the complement 3 glomerulopathy is refractory to other treatment.

31. The method of claim 1, the human has refractory disease to immunosuppressive drugs.

32. The method of claim 1, wherein the human has refractory disease to one or more of rituximab, cyclophosphamide, mycophenolate mofetil, tacrolimus, and steroids.

33. The method of claim 1, wherein the compound is administered orally.

34. The method of claim 1, wherein the compound is administered twice daily.

35. The method of claim 1, wherein the human receives 30 mg of the compound twice daily.

36.-41. (canceled)
